(19) United States
(12) Patent Application Publication
(10) Pub. No.: US 2007/0244075 A1
(43) Pub. Date: Oct. 18, 2007

(54) Isoflavonoid Prodrugs, Compositons Thereof and Therapeutic Methods Involving Same

(75) Inventors: Andrew Heaton, Abbotsford (AU); Graham Edmund Kelly, Northbridge (AU)

Correspondence Address: SUGHRUE MION, PLLC, 2100 PENNSYLVANIA AVENUE, N.W., SUITE 800, WASHINGTON, DC, 20037, US

(73) Assignee: NOVOGEN RESEARCH PTY LTD

(21) Appl. No.: 10/579,789

(22) PCT Filed: Nov. 18, 2004
(86) PCT No.: PCT/AU04/01602

§ 371 Date: Jan. 19, 2007

(30) Foreign Application Priority Data
Nov. 18, 2003 (AU) 2003906353

(51) Int. Cl. A61K 31/662; A61P 15/00; C07F 9/09

(52) U.S. Cl. 514/99; 426/590; 549/222

(57) ABSTRACT

Phosphate esters of isoflavonoid compounds are prepared for use as prodrugs, medicaments and in formulations, drinks and foodstuffs.

### FIELD OF THE INVENTION

This invention relates to compounds, formulations, drinks, foodstuffs, methods and therapeutic uses involving, containing, comprising, including and/or for preparing certain isoflavene prodrugs and analogues thereof. In particular, the invention relates to phosphate esters of isoflavonoids and derivatives, medicaments involving same and therapeutic uses thereof.

### BACKGROUND

Isoflavones and many derivatives thereof possess a very wide range of important biological properties including oestrogenic effects. Isoflavones such as genistein and daidzein have been shown to be involved in the modulation or attenuation of levels of estrogenic steroids in the body. More recently, isoflavenes and in particular dehydroequol have been shown to possess strong chemotherapeutic properties. In some areas of biological activity, there are even some contradictions, for example, some isoflavonoids act as agonists of the estrogen receptor while others act as antagonists of the estrogen receptor. It is believed that there is a strong correlation between lowering levels of biologically active estrogenic steroids in the body with lower incidences of cancer such as breast cancer and many other diseases and conditions.

However, the biological activity of isoflavonoids in animals is not conserved across the spectrum of the isoflavonoid family and therefore cannot be predicted, especially where bioavailability is involved. Thus each specific structural variation of the basic isoflavonoid molecule can yield a highly individual biological profile in animals ranging from nil effect through to potent effect. Furthermore, it is thought that some conjugates of biologically active molecules, such as phosphate esters of some biologically active estrogenic steroids, can be largely inactive.

There is a strong need to identify new, improved, better and/or alternative pharmaceutical compositions and agents for the treatment, amelioration and prevention of diseases, conditions and disorders. There is a further need to provide new isoflavonoid compounds and derivatives for the improved formulation, bioavailability and delivery of these compounds. There is also a need for new and different therapies to be available to both physicians and the general public to combat the numerous and various types of diseases and disorders which affect members of the population.

A requirement accordingly exists for the provision of new isoflavonoid compounds and derivatives thereof which are therapeutically beneficial and which show improved, alternative or at least comparable bioactive and bioavailable properties to that of known isoflavonoid compounds.

### SUMMARY OF THE INVENTION

The present inventors have surprisingly found that phosphate esters of isoflavonoid compounds show good aqueous solubility and bioavailability and exhibit beneficial biological properties. In particular phosphate esters when administered will exhibit a wide range of therapeutic activities including the ability to address oestrogen-levels in the body.

Whilst not wishing to be limited to theory, it is believed that isoflavene prodrugs and derivatives thereof, and in particular isoflavonoid phosphate esters the invention will result in the reduction in the supply of estrogenic steroids, reducing the risk or severity of oestrogen-related diseases and conditions. It is also thought that the isoflavonoid phosphate esters of the invention will provide for the regulation of a range of molecular targets in mammalian cells. Typically, these molecular targets are intimately involved in signal transduction processes that are fundamental to critical cellular processes such as cell growth, differentiation, migration, and death. It can be seen therefore that these surprising biochemical effects have broad and important implications for the health of animals including humans. These and other preferred objects of the invention are described herein.

Thus, according to an aspect of the invention there is provided an isoflavonoid phosphate ester compound of the general formula I: 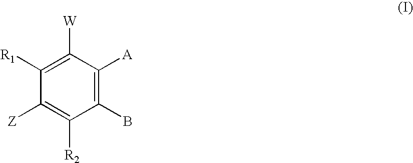
in which
R₁, R₂ and Z are independently M₂PO₄—, hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 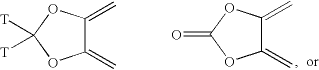
R₁ is as previously defined, and R₂ and Z taken together with the carbon atoms to which they are attached form a five-membered ring selected from 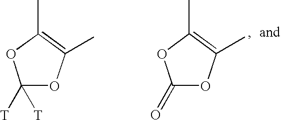
W is R₁, and A and B taken together with the carbon atoms to which they are attached form a six-membered ring selected from 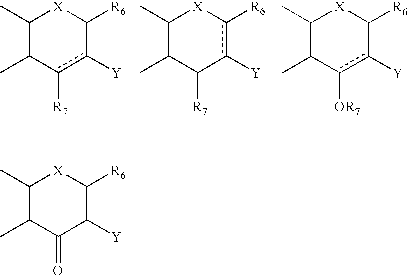
   wherein
R₃ is hydrogen, alkyl, aryl, arylalkyl, an amino acid, C(O)R₁₁ where R₁₁ is hydrogen alkyl, aryl, arylalkyl or an amino acid, or CO₂R₁₂ where R₁₂ is hydrogen, alkyl, haloalkyl, aryl, heteroaryl or arylalkyl,
R₄ is hydrogen, alkyl or aryl,
or R₃ and R₄ taken together with the nitrogen to which they are attached comprise pyrrolidinyl or piperidinyl,
R₅ is M₂PO₄—, hydrogen, C(O)R₁₁ where R₁₁ is as previously defined, or CO₂R₁₂ where R₁₂ is as previously defined,
R₆ is M₂PO₄—, hydrogen, hydroxy, alkyl, aryl, amino, thio, NR₃R₄, COR₁₁ where R₁₁ is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl,
R₈ is M₂PO₄—, hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀ is hydrogen, alkyl, haloalkyl, amino, aryl, arylalkyl, an amino acid, alkylamino or dialkylamino,
the drawing “ [symbol] ” represents either a single bond or a double bond,
M is independently hydrogen, a straight or branched alkyl, alkenyl, alkynyl, alkoxyalkyl, alkylthioalkyl, or aminoalkyl, a substituted or non-substituted cycloalkyl, an aryl, aralkyl, or alkylaryl, and a substituted cycloalkyl where at least one ring contains one or more of a nitrogen, sulfur, oxygen, phoshorous or silicon heteroatom in the at least one ring;
T is independently hydrogen, alkyl or aryl,
X is O, NR₄ or S, preferably O, and
Y is 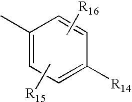
   wherein
R₁₄, R₁₅ and R₁₆ are independently M₂PO₄—, hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo, and
   wherein at least one of R₁, R₂, R₅, R₆, R₈, R₁₄, R₁₅, R₁₆, Z, W or A where present is independently M₂PO₄—,
   or a pharmaceutically acceptable salt thereof.

In a preferred embodiment, the phosphate ester moiety may be present as the corresponding salt —O—PO(OM)₂, where M is hydrogen or a pharmaceutically acceptable counter ion, more preferably Na⁺, K⁺, Li⁺, Mg⁺⁺ or NH₃⁺, more preferably Na⁺.

It has surprisingly been found by the inventors that compounds of the general formula I: 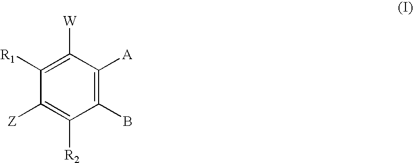
in which

R₁, R₂, W, A, B and Z are as defined above have particular utility and effectiveness in the treatment, prophylaxis, amelioration defence against, and/or prevention of the following diseases and disorders (for convenience hereinafter referred to as the “therapeutic indications”):
(a) all forms of cancer (pre-malignant, benign and malignant) in all tissues of the body. In this regard, the compounds may be used as the sole form of anti-cancer therapy or in combination with other forms of anti-cancer therapy including but not limited to radiotherapy and chemotherapy;
(b) diseases and disorders associated with inflammatory reactions of an abnormal or prolonged nature in any of the body's tissues including but not limited to rheumatoid arthritis, tendonitis, inflammatory bowel disease, ulcerative colitis, Crohn's Disease, sclerosing cholangitis;
(c) papulonodular skin lesions including but not limited to sarcoidosis, angiosarcoma, Kaposi's sarcome, Fabry's Disease
(d) papulosquamous skin lesions including but not limited to psoriasis, Bowen's Disease, and Reiter's Disease;
(e) actinic damage characterized by degenerative changes in the skin including but not limited to solar keratosis, photosensitivity diseases, and wrinkling;
(f) diseases and disorders associated with abnormal angiogenesis affecting any tissue within the body including but not limited to hemangiomas and telangiectasia;
(g) proliferative disorders of bone marrow including but not limited to megaloblastic disease, myelodysplastic syndromes, polycythemia vera, thrombocytosis and myelofibrosis;
(h) autoimmune disease characterized by abnormal immunological responses including but not limited to multiple sclerosis, Type 1 diabetes, systemic lupus erythematosis, and biliary cirrhosis;
(i) neurodegenerative diseases and disorders characterized by degenerative changes in the structure of the neurological system including but not limited to Parkinson's Disease, Alzheimer's Disease, muscular dystrophy, Lou-Gehrig Disease, motorneurone disease;
(j) diseases and disorders associated with degenerative changes within the walls of blood vessels including but not limited to atherosclerosis, atheroma, coronary artery disease, stroke, myocardial infarction, hypertensive vascular disease, malignant hypertension, thromboangiitis obliterans, fibromuscular dysplasia;
(k) diseases and disorders associated with abnormal immunological esponses including but limited to dermatomyositis and scleroderma;
(l) diseases and disorders associated with degenerative changes within the eye including but not limited to cataracts, macular degeneration, retinal atrophy.

In particular the isoflavene compounds also surprisingly have been found to have a potent effect on the production and function of reproductive hormones such as estrogens and androgens. As a result of this, these compounds may be used in the treatment and prevention of the following disorders and diseases:
(a) conditions in women associated with abnormal estrogen/androgen balance including but not limited to cyclical mastalgia, acne, dysmenorrhoea, uterine fibroids, endometriosis, ovarian cysts, premenstrual syndrome, acute menopause symptoms, osteoporosis, senile dementia, infertility; and
(b) conditions in men associated with abnormal estrogen/androgen balance including but not limited to benign prostatic hypertrophy, infertility, gynecomastia, alopecia hereditaria and various other forms of baldness.

Thus according to another aspect of the present invention there is provided a method for the treatment, prophylaxis, amelioration, defence against, and/or prevention of one or more of the therapeutic indications which comprises administering to a subject a therapeutically effective amount of one or more compounds of formula I as defined above.

According to another aspect of the present invention there is provided the use of compounds of formula I for the manufacture of a medicament for the treatment, amelioration, defence against, prophylaxis and/or prevention of one or more of the therapeutic indications.

According to another aspect of the present invention there is provided the use of one or more compounds of formula I in the treatment, amelioration, defence against, prophylaxis and/or prevention of one or more of the therapeutic indications.

According to another aspect of the present invention there is provided an agent for the treatment, prophylaxis, amelioration, defence against and/or treatment of the therapeutic indications which comprises one or more compounds of formula I either alone or in association with one or more carriers or excipients.

According to another aspect of the present invention there is provided a therapeutic composition which comprises one or more compounds of formula I in association with one or more pharmaceutical carriers and/or excipients.

According to another aspect of the present invention there is provided a drink or food-stuff, which contains one or more compounds of formula I.

According to another aspect of the present invention there is provided a microbial culture or a food-stuff containing one or more microbial strains which microorganisms produce one or more compounds of formula I.

According to another aspect of the present invention there is provided one or more microorganisms which produce one or more compounds of formula I. Preferably the microorganism is a purified culture, which may be admixed and/or administered with one or more other cultures which product compounds of formula I.

Throughout this specification and the claims which follow, unless the text requires otherwise, the word “comprise”, and variations such as “comprises” or “comprising”, will be understood to imply the inclusion of a stated integer or step or group of integers or steps but not the exclusion of any other integer or step or group of integers or steps.

### BRIEF DESCRIPTION OF THE DRAWINGS

FIG. 1 depicts pharmacokinetic data comparing free and total dehydroequol concentrations in serum from mice injected i.p with bolus dosages of DHE bisphosphate prepared in PBS and dosed at 25 mg/kg.

FIGS. 2a and 2b depict pharmacokinetic data comparing free and total dehydroequol concentrations in serum from mice injected i.p with bolus dosages of dehydroequol prepared in different formulations. DHE bisphosphate formulations were prepared in PBS and dosed at 25 mg/kg. DHE PEG:PBS formulations were prepared in a 1:1 PEG:PBS formulations and dosed at 50 mg/kg. DHE-HPBCD formulations were prepared in 20% HPBCD (HPBCD prepared in PBS) and dosed at 50 mg/kg (total DHE levels not shown).

### DETAILED DESCRIPTION OF THE INVENTION

The term “isoflavonoid” is generally taken to mean ring-fused benzopyran molecules having a pendent phenyl group from the pyran ring based on a 1,2-diphenylpropane system. Thus, the classes of compounds generally referred to as isoflavones, isoflavenes, isoflavans, isoflavanones, isoflavanols and the like are generically referred to herein as isoflavonoids, isoflavonoid compounds, or isoflavone metabolites or derivatives thereof.

Preferred isoflavonoid compounds of invention are the isoflavan-4-ones, isoflavenes, isoflavan-4-ols and isoflavans, which in general are hydrogenated products from the base isoflavones, which compounds may also be optionally substituted.

The term “alkyl” is taken to include straight chain, branched chain and cyclic (in the case of 5 carbons or greater) saturated alkyl groups of 1 to 10 carbon atoms, preferably from 1 to 6 carbon atoms, such as methyl, ethyl, propyl, isopropyl, butyl, isobutyl, secbutyl, tertiary butyl, pentyl, cyclopentyl, and the like. The alkyl group is more preferably methyl, ethyl, propyl or isopropyl. The alkyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkenyl” is taken to include straight chain, branched chain and cyclic (in the case of 5 carbons or greater) hydrocarbons of 2 to 10 carbon atoms, preferably 2 to 6 carbon atoms, with at lease one double bond such as ethenyl, 1-propenyl, 2-propenyl, 1-butenyl, 2-butenyl, 2-methyl-1-peopenyl, 2-methyl-2-propenyl, and the like. The alkenyl group is more preferably ethenyl, 1-propenyl or 2-propenyl. The alkenyl groups may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “alkynyl” is taken to include both straight chain and branched chain hydrocarbons of 2 to 10 carbon atoms, preferably 2 to 6 carbon atoms, with at least one triple bond such as ethynyl, 1-propynyl, 2-propynyl, 1-butynyl, 2-butynyl, and the like. The alkynyl group is more preferably ethynyl, 1-propynyl or 2-propynyl. The alkynyl group may optionally be substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl.

The term “aryl” is taken to include phenyl, biphenyl and naphthyl and may be optionally substituted by one or more C₁-C₄-alkyl, hydroxy, C₁-C₄-alkoxy, carbonyl, C₁-C₄-alkoxycarbonyl, C₁-C₄-alkylcarbonyloxy or halo.

The term “heteroaryl” is taken to include five-membered and six-membered rings which include at least one oxygen, sulfur or nitrogen in the ring, which rings may be optionally fused to other aryl or heteroaryl rings including but not limited to furyl, pyridyl, pyrimidyl, thienyl, imidazolyl, tetrazolyl, pyrazinyl, benzofuranyl, benzothiophenyl, quinolyl, isopuinolyl, purinyl, morpholinyl, oxazolyl, thiazolyl, pyrrolyl, xanthinyl, purine, thymine, cytosine, uracil, and isoxazolyl. The heteroaromatic group can be optionally substituted by one or more of fluorine, chlorine, bromine, iodine, carboxyl, C₁-C₄-alkoxycarbonyl, C₁-C₄-alkylamino-carbonyl, di-(C₁-C₄-alkyl)-amino-carbonyl, hydroxyl, C₁-C₄-alkoxy, formyloxy, C₁-C₄-alkyl-carbonyloxy, C₁-C₄-alkylthio, C₃-C₆-cycloalkyl or phenyl. The heteroaromatic can be partially or totally hydrogenated as desired.

The term “halo” is taken to include fluoro, chloro, bromo and iodo, preferably fluoro and chloro, more preferably fluoro. Reference to for example “haloalkyl” will include monohalogenated, dihalogenated and up to perhalogenated alkyl groups. Preferred haloalkyl groups are trifluoromethyl and pentafluoroethyl.

The term “pharmaceutically acceptable salt” refers to an organic or inorganic moiety that carries a charge and that can be administered in association with a pharmaceutical agent, for example, as a counter-cation or counter-anion in a salt. Pharmaceutically acceptable cations, which include the moiety M, are known to those of skilled in the art, and include but are not limited to sodium, potassium, calcium, zinc and quaternary amine. Pharmaceutically acceptable anions are known to those of skill in the art, and include but are not limited to chloride, acetate, citrate, bicarbonate and carbonate.

The term “pharmaceutically acceptable derivative” or “prodrug” refers to a derivative of the active compound that upon administration to the recipient is capable of providing directly or indirectly, the parent compound or metabolite, or that exhibits activity itself.

As used herein, the terms “treatment”, “prophylaxis” or “prevention”, “amelioration” and the like are to be considered in their broadest context. In particular, the term “treatment” does not necessarily imply that an animal is treated until total recovery. Accordingly, “treatment” includes amelioration of the symptoms or severity of a particular condition or preventing or otherwise reducing the risk of developing a particular condition.

The invention in particular relates to the compounds of the general formula II and uses thereof: 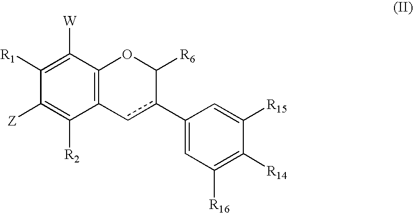
in which

R₁, R₂, R₅, R₆, R₁₄, R₁₅, W and Z are as defined above, the drawing “ [symbol] ” represents either a single bond or a double bond, and more preferably where

the drawing “ [symbol] ” represents a double bond.

In another aspect, the invention in particular relates to the compounds of the general formula III and uses thereof:

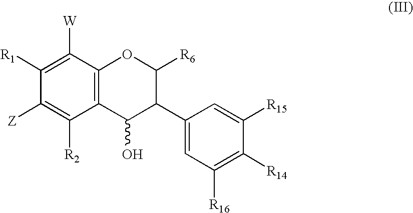

in which

R₁, R₂, R₅, R₆, R₁₄, R₁₅, W and Z are as defined above.

In another aspect, the invention in particular relates to the compounds of the general formula IV and uses thereof:

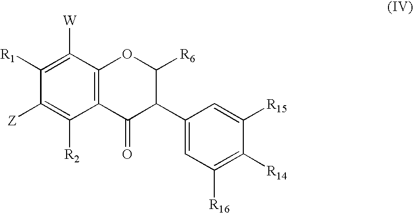

in which

R₁, R₂, R₅, R₆, R₁₄, R₁₅, W and Z are as defined above.

Particularly preferred compounds of the present invention are the isoflavonoid compounds as follows:
Isoflavonoid-O—PO(OM)₂

wherein M is independently hydrogen or a counter cation, and

wherein the isoflavene compound or derivative is mono-, di-, or per-phosphorylated and may be derived from the following hydroxyl-containing isoflavanone, isoflavene, isoflavanol and isoflavan compounds and derivatives 1-22 as follows:

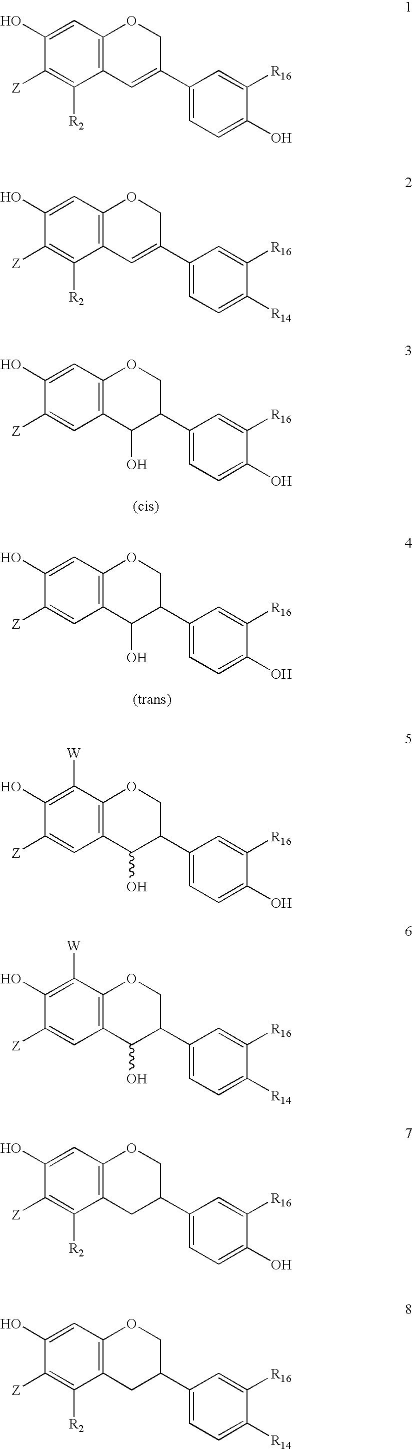

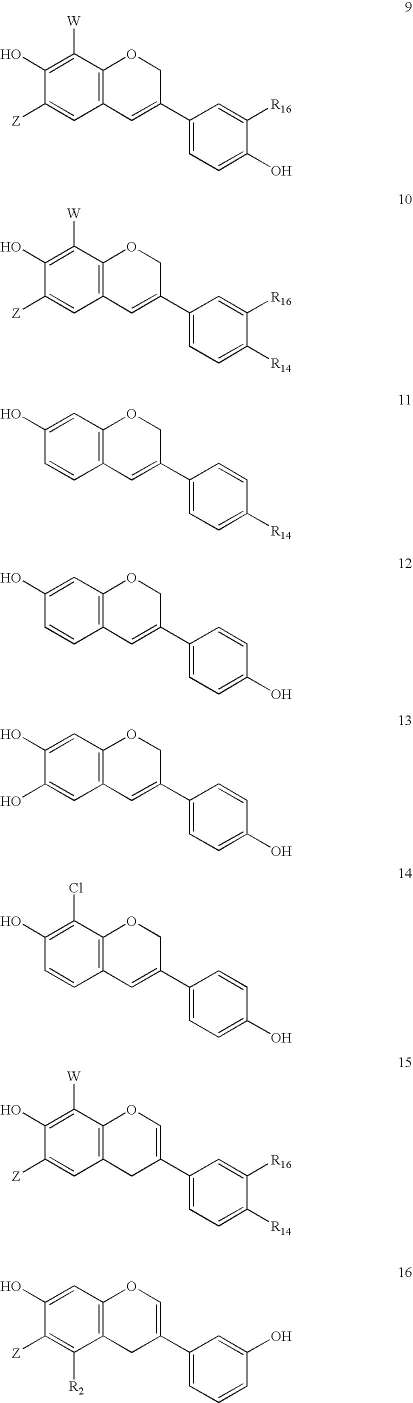

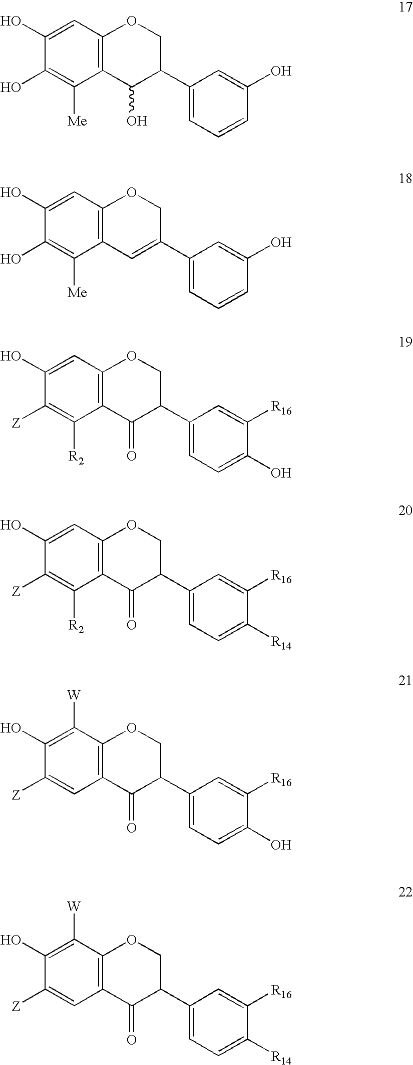

wherein

R₂, R₁₆, W and Z are independently H, OH, Cl, Br, Me or OMe, and

R₁₄ is H, OMe, Me, Cl or Br.

In a most preferred embodiment isoflavonoid compound or derivative is a novel mono-, di- or per-phosphate ester of dihydrodaidzein, dihydrogenestein, tetrahydrodaidzein, dehydroequol or equol, most preferably is a phosphate ester of dehydroequol.

Compounds of the present invention have particular application in the treatment of diseases associated with or resulting from estrogenic effects, androgenic effects, vasodilatory and spasmodic effects, inflammatory effects and oxidative effects.

The amount of one or more compounds of formula I which is required in a therapeutic treatment according to the invention will depend upon a number of factors, which include the specific application, the nature of the particular compound used, the condition being treated, the mode of administration and the condition of the patient. Compounds of formula I may be administered in a manner and amount as is conventionally practised. See, for example, Goodman and Gilman, The Pharmacological Basis of Therapeutics, 1299 (7th Edition, 1985). The specific dosage utilised will depend upon the condition being treated, the state of the subject, the route of administration and other well known factors as indicated above. In general, a daily dose per patient may be in the range of 0.1 mg to 2 g; typically from 0.5 mg to 1 g; preferably from 50 mg to 200 mg. The length of dosing may range from a single dose given once every day or two, to twice or thrice daily doses given over the course of from a week to many months to many years as required, depending on the severity of the condition to be treated or alleviated. It will be further understood that for any particular subject, specific dosage regimens should be adjust over time according to the individual need and the professional judgment of the person administering or supervising the administration of the compositions.

The production of pharmaceutical compositions for the treatment of the therapeutic indications herein described are typically prepared by admixture of the compounds of the invention (for convenience hereafter referred to as the “active compounds”) with one or more pharmaceutically or veterinarially acceptable carriers and/or excipients as are well known in the art.

The carrier must, of course, be acceptable in the sense of being compatible with any other ingredients in the formulation and must not be deleterious to the subject. The carrier or excipient may be a solid or a liquid, or both, and is preferably formulated with the compound as a unit-dose, for example, a tablet, which may contain from 0.5% to 59% by weight of the active compound, or up to 100% by weight of the active compound. One or more active compounds may be incorporated in the formulations of the invention, which may be prepared by any of the well known techniques of pharmacy consisting essentially of admixing the components, optionally including one or more accessory ingredients.

The formulations of the invention include those suitable for oral, rectal, optical, buccal (for example, sublingual), parenteral (for example, subcutaneous, intramuscular, intradermal, or intravenous) and transdermal administration, although the most suitable route in any given case will depend on the nature and severity of the condition being treated and on the nature of the particular active compound which is being used.

Formulation suitable for oral administration may be presented in discrete units, such as capsules, sachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture such as to form a unit dosage. For example, a tablet may be prepared by compressing or moulding a powder or granules containing the active compound, optionally with one or more accessory ingredients. Compressed tablets may be prepared by compressing, in a suitable machine, the compound of the free-flowing, such as a powder or granules optionally mixed with a binder, lubricant, inert diluent, and/or surface active/dispersing agent(s). Moulded tablets may be made by moulding, in a suitable machine, the powdered compound moistened with an inert liquid binder.

Formulations suitable for buccal (sublingual) administration include lozenges comprising the active compound in a flavoured base, usually sucrose and acacia or tragacanth; and pastilles comprising the compound in an inert base such as gelatin and glycerin or sucrose and acacia.

Compositions of the present invention suitable for parenteral administration conveniently comprise sterile aqueous preparations of the active compounds, which preparations are preferably isotonic with the blood of the intended recipient. These preparations are preferably administered intravenously, although administration may also be effected by means of subcutaneous, intramuscular, or intradermal injection. Such preparations may conveniently be prepared by admixing the compound with water or a glycine buffer and rendering the resulting solution sterile and isotonic with the blood. Injectable formulations according to the invention generally contain from 0.1% to 60% w/v of active compound and are administered at a rate of 0.1 ml/minute/kg.

Formulations suitable for rectal or vaginal administration are preferably presented as unit dose suppositories. These may be prepared by admixing the active compound with one or more conventional solid carriers, for example, cocoa butter, and then shaping the resulting mixture.

Formulations or compositions suitable for topical administration to the skin preferably take the form of an ointment, cream, lotion, paste, gel, spray, aerosol, or oil. Carriers which may be used include Vaseline, lanoline, polyethylene glycols, alcohols, and combination of two or more thereof. The active compound is generally present at a concentration of from 0.1% to 0.5% w/w, for example, from 0.5% to 2% w/w. Examples of such compositions include cosmetic skin creams.

Formulations suitable for transdermal administration may be presented as discrete patches adapted to remain in intimate contact with the epidermis of the recipient for a prolonged period of time. Such patches suitably contain the active compound as an optionally buffered aqueous solution of, for example, 0.1 M to 0.2 M concentration with respect to the said active compound.

Formulations suitable for transdermal administration may also be delivered by iontophoresis (see, for example, Pharmaceutical Research 3 (6), 318 (1986)) and typically take the form of an optionally buffered aqueous solution of the active compound. Suitable formulations comprise citrate or bis/tris buffer (pH 6) or ethanol/water and contain from 0.1 M to 0.2 M active ingredient.

Formulations suitable for inhalation may be delivered as a spray composition in the form of a solution, suspension or emulsion. The inhalation spray composition may further comprise a pharmaceutically acceptable propellant such as carbon dioxide or nitrous oxide.

The active compounds may be provided in the form of food stuffs, such as being added to, admixed into, coated, combined or otherwise added to a food stuff. The term food stuff is used in its widest possible sense and includes liquid formulations such as drinks including dairy products and other foods, such as health bars, desserts, etc. Food formulations containing compounds of the invention can be readily prepared according to standard practices.

Compounds of the present invention have potent antioxidant activity and thus find wide application in pharmaceutical and veterinary uses, in cosmetics such as skin creams to prevent skin ageing, in sun screens, in foods, health drinks, shampoos, and the like.

It has surprisingly been found that compounds of the formula I interact synergisticly with vitamin E to protect lipids, proteins and other biological molecules from oxidation.

Accordingly a further aspect of this invention provides a composition comprising one or more compounds of formula I, vitamin E, and optionally a pharmaceutically, veterinarily or cosmetically acceptable carriers and/or excipients.

Therapeutic methods, uses and compositions may be for administration to humans or animals, such as companion and domestic animals (such as dogs and cats), birds (such as chickens, turkeys, ducks), livestock animals (such as cattle, sheep, pigs and goats), for use in aquaculture applications and the like.

The isoflavonoid prodrugs and derivatives can also be co-administered with other active materials that do not impair the desired action, or with materials that supplement the desired action, such as antibiotics, antifungals, antiinflammatories, or antiviral compounds.

The active agent can comprise two or more isoflavones or derivatives thereof in combination or synergistic mixture. The active compounds can also be administered with lipid lowering agents such as probucol and nicotinic acid; platelet aggregation inhibitors such as aspirin; antithrombotic agents such as coumadin; calcium channel blockers such as verapamil, diltiazem, and nifedipine; angiotensin converting enzyme (ACE) inhibitors such as captopril and enalapril, and β-blockers such as propanolol, terbutalol, and labetalol. The compounds can also be administered in combination with nonsteriodal antiinflammatories such as ibuprofen, indomethacin, aspirin, fenoprofen, mefenamic acid, flufenamic acid and sulindac. The compounds can also be administered with corticosteroids.

The co-administration may be simultaneous or sequential. Simultaneous administration may be effected by the compounds being in the same unit dose, or in individual and discrete unit doses administered at the same or similar time. Sequential administration may be in any order as required and typically will require an ongoing physiological effect of the first or initial active agent to be current when the second or later active agent is administered, especially where a cumulative or synergistic effect is desired.

Isoflavone compounds are suitable starting materials for the synthesis of the isoflavonoid compounds of formula I and these isoflavone starting materials may be prepared by standard methods known to those skilled in the art. Suitable methods may be found in, for example, International Patent Applications WO 98/08503 and WO 00/49009 which are incorporated herein in their entirety by reference. Chemical functional group protection, deprotection, synthons and other techniques known to those skilled in the art may be used where appropriate in the synthesis of the compounds of the present invention. Derivatisation of the hydroxy substituted isoflavones to form the conjugates of the present invention may be performed by any suitable method as known to one skilled in the art.

The isoflavone starting materials may also be obtained in the form of concentrates or extracts from plant sources. Again, those skilled in the art will readily be able to identify suitable plant species, however, for example, plants of particular utility include leguminous plants. More preferably, the isoflavone extract may be obtained from obtained from chickpea, lentils, beans, red clover or subterranean clover species and the like.

The aqueous solubility of isoflavonoids is important for their formulation into pharmaceuticals, foodstuffs and cosmetics, many of which are aqueous-based systems. Low solubility is also frequently an impediment to efficient bioavailability in orally administered products. Low solubility is a particularly serious impediment to formulation of intravenous medications, which are most often delivered in aqueous media. The isoflavonoid phosphate esters of the invention are presented in forms which have increased bioavailability, especially enhanced aqueous solubility relative to the unmodified compounds, while substantially retaining the active properties of such unmodified compounds. The phosphate ester is useful as a pro-drug having a polar (solubilising) leaving group which can be readily hydrolysed under physiological conditions to produce the corresponding isoflavonoid compound.

In preferred embodiments, an alcohol functionality of an isoflavonoid is esterified using a phosphoric acid group yielding a phosphate ester. In general, fluids of the digestive and absorptive gastrointestinal tract, other acids, and various enzymes are capable of hydrolysing the esterified isoflavonoid to the starting isoflavonoid.

The phosphate ester is preferably a (OH)₂PO₂ group due to the presence of the two polar groups, and that it is a good solubliser and has high biological compatibility. Where the M group for M₂PO-4- is not hydrogen, it would generally be expected that the solubility would be less for the compound and would therefore be less favoured. Where M is an alkyl group, for example, the non-polar group is preferably selected to be small.

It is also contemplated to employ metal salt complexes of the esterified isoflavones, especially Li⁺, Na⁺, K⁺, Mg⁺⁺ and ammonium salts, including NH₄⁺ and low molecular weight mono- or polyalkylammonium counter ions.

The examples that follow are not considered to limit the invention as described.

### EXAMPLES

The isoflavonoid phosphate esters of the invention may be prepared by standard chemical processes known by those skilled in the art from available starting materials and straight forward synthetic methods. In this way, several embodiments of the inventive subject matter can be prepared and characterised. These examples all fall within the group of pro-compounds of formula I having at least one group of the formula M₂PO₄—. These new phosphate esters are all water soluble and readily hydrolysed in vivo, yet are generally quite stable in aqueous solutions in vitro at normal pH at ambient or body temperature, and are more stable as solids.

### Example 1

### Phosphate Esters of Dehydroequol

A solution of dehydroequol (120 mg, 0.5 mmole) and di-tert-butyl phosphoramidite (330 ul, 1.0 mmole) in DMF (1 ml) is stirred under argon while 1H-tetrazole (210 mg in 0.5 ml of DMF; 3.0 mmole) is added dropwise. The solution is cooled to −20° C., then a solution of m-chloroperbenzoic acid (260 mg in 0.5 ml of methylene chloride, 1.5 mmole) is added dropwise. After warming to room temperature, the mixture is diluted threefold with ethyl acetate, then washed with 10% sodium metabisulphite and 10% sodium bicarbonate.

The ethyl acetate solution, containing the butyl esters of the dehydroequol phosphates, is washed with 1M HCl and dried over sodium sulfate. After removal of the solvent in vacuo, the residue is treated with 30% TFA in acetic acid for 90 minutes at room temperature. The solvents are removed in vacuo, and the residue is taken up in ethanol and neutralised with sodium hydroxide to pH 5.5. Removal of the solvent in vacuo affords a mixture of sodium salts of dehydroequol phosphates, 130 mg.

Analysis of the phosphate mixture indicated the presence of the 4′-phosphate, the 7-phosphate and the 4′,7-diphosphate derivatives. Where esterification of the compounds of the invention affords mixtures of phosphate esters, they may be separated into individual components by standard separation techniques including fractional crystallisation, column chromatography and HPLC.

The isoflavonoid phosphate esters prepared by the above methods include:

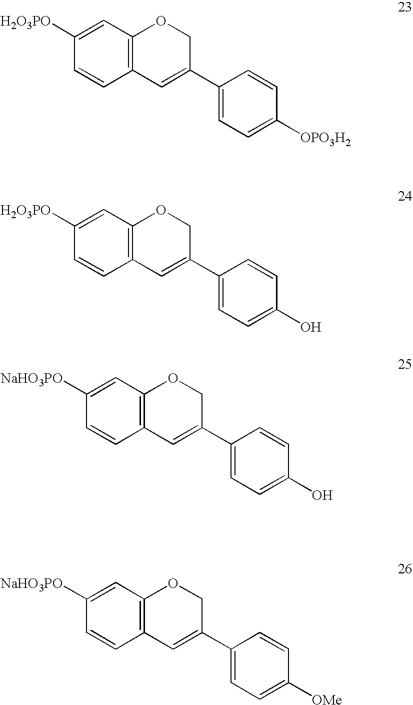

Likewise, phosphate esters of dihydrodaidzein, tetrahydrodaidzein and equol were synthesised affording the following compounds.

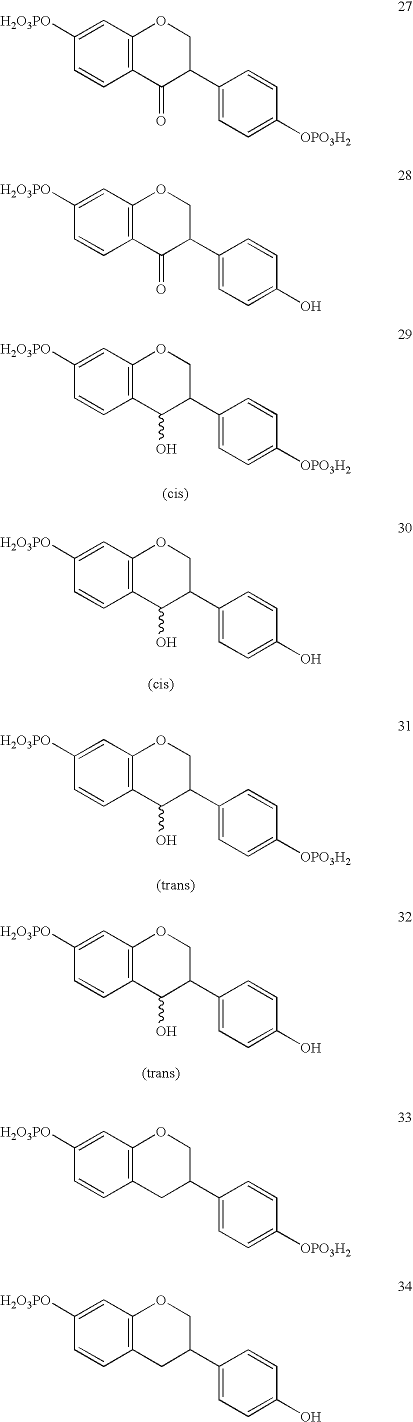

and pharmaceutically acceptable salts thereof

### Example 2

### Dehydroequol- 7-phosphate

Dehydroequol with its hydroxy group protected at the pendant phenyl 4′-position undergoes reaction according to Example 1 to afford the corresponding 7-phosphate derivative. Any suitable protective group may be employed including MOM or MEM ethers and benzylic ethers. These groups optionally may be removed after phosphorylation. The protecting groups where used may be incorporated in the synthesis of the isoflavonoid starting materials following any of the methods referred to herein, or may be attached at a later time by taking advantage of synthons, chemical reactivity, polarity, electronic considerations, or steric conditions on or near any of the target hydroxy groups.

By these methods mono- di- and per-phosphorylated derivatives of compounds 1-22 described herein are synthesised. The phosphorus acids and pharmaceutically acceptable salts thereof are thus prepared. Proton or carbon magnetic resonance spectra, IR and/or mass spectra was used to characterise the compounds synthesised.

### Example 3

The bioavailability of the isoflavonoid phosphoric esters of the invention are tested by the in vitro hydrolysis of the dehydroequol phosphates by various enzymes and biological media. Results are determined by measuring the amount of free dehydroequol by HPLC. The sera and media used include human serum, human blood, rat blood, alkaline phosphatase type VII-S (bovine intestinal mucosa) and alkaline phosphatase type XXIV (human placenta).

The bioavailability and conversion rate from the ester depends on a number of factors including the nature of the phosphate ester and substitutions thereon, the media, any enzymes present, the temperature and pH. By controlling these various parameters, it is found that some degree of regulation or control can be obtained by altering the half-life of the ester prodrug to better match the desired bioavailability rate.

### Example 4

The esterified isoflavonoids are found to be readily converted to free isoflavonoids in biological media such as gastrointestinal fluid and blood. Among other things, gastrointestinal fluids often have enzymes and sufficiently high pH to hydrolyse ester bonds, and blood generally contains enzymes such as phosphatases which can hydrolyse phosphate ester bonds.

### Pharmacokinetic Experiments

Two separate PK experiments were conducted using dehydroequol (DHE)-bisphosphate formulated in PBS by i.p. and oral modes of delivery. Three animals were to be allocated per timepoint with 5 timepoints (15 min, 30 min, 1 hr, 4 hr and 24 hr) (15 mice per study).

The aim was to determine whether the PK profile was comparable when delivered i.p. vs oral.

Protocol—i.p. Administration
1. Female nude mice were maintained on an isoflavone free diet for at least one week to remove background isoflavone levels in plasma.
2. On day prior to experimentation, 3 mice were assigned per time-point and marked with unique identifiers. Each mouse was weighed to determine the density of DHE bisphosphate required per i.p. injection to achieve a dose of 50 mg/kg for each mouse.
   A slight excess of formulated DHE-bisphosphate was prepared and the mass of powder adjusted accordingly. The remaining solution was stored at −20° C. for QA analysis.
3. Each mouse was injected into the lower right or left quadrant of the abdomen, ensuring that the needle was not in a vessel or loop of bowel. Once the DHE-boisphosphate was administered, the mice were placed in a cage until each time point (15 min, 30 min, 1 hr, 4hr, 24 hr).
4. Each moused was killed by cervical dislocation, then the blood collected via the thoracic cavity as per SOP BD-009 using a 20 gauge needle.
5. The blood was allowed to clot then centrifuged at top speed for 3 minutes using a bench-top mini-microfuge at RT.
6. Serum was aspirated into an appropriately labelled eppendorf tube and stored at −20° C. until analysed. Sera from animals dosed with vehicle control and formulated DHE-bisphosphate were stored at −20° C. along with 200 ul aliquots of the vehicle and formulated DHE-bisphosphate for analysis.

Protocol—Oral Administration
1. Female BALB/c mice were maintained on an isoflavone free diet for at least one week to remove background isoflavone levels in plasma.
2. On day prior to experimentation, 3 mice were assigned per time-point and marked with unique identifiers. Each mouse was weighed to determine the density of DHE-bisphosphate required to dose animals at 50 mg/kg.
3. Each mouse was restrained and gavaged an appropriate volume of formulated DHE-bisphosphate to achieve a dose of 50 mg/kg. Once DHE-bisphosphate was administered, the mice were placed in a cage until time point (15 min, 30 min, 1 hr, 4 hr, 24 hr). The control animals were gavaged with 200 μl 1% CMC control. Control animals were culled at 15 min, 30 min, 1 hr, 4 hr, 24 hr timepoints.
4. At the designated time points, each mouse was killed by cervical dislocation, then the blood collected via the thoracic cavity as per SOP BD-009 using a 20 gauge needle.
5. The blood was allowed to clot then centrifuged at top speed for 3 minutes using a bench-top mini-microfuge at RT.
6. Serum was aspirated into an appropriately labelled eppendorf tube and stored at −20° C. until analysed. Sera from animals dosed with vehicle control and formulated DHE-bisphosphate were stored at −20° C. along with 200 ul aliquots of the vehicle and formulated DHE-bisphosphate for analysis.
7. The remaining three animals were gavaged with formulation vehicle at time zero and culled at time 30 min. Serum was stored with the other samples.

### RESULTS

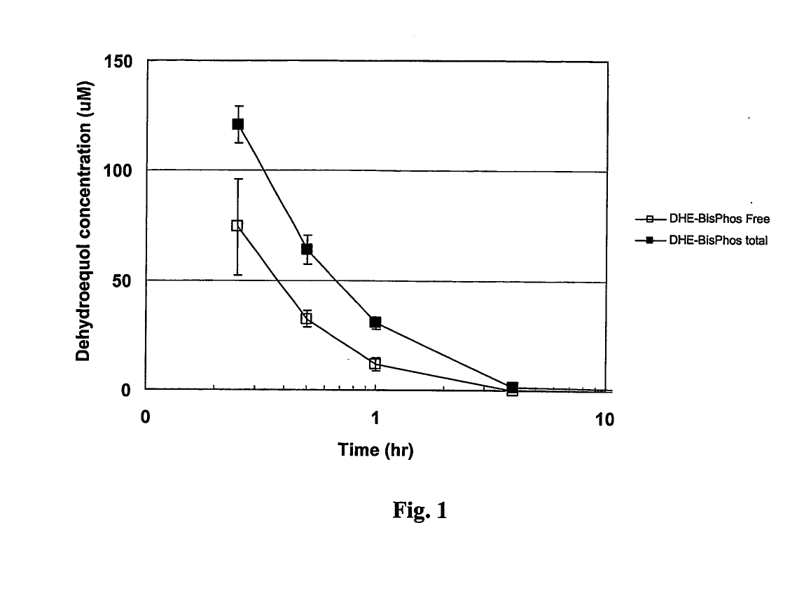

When dosed at 25 mg/kg in mice the DHE-bisphosphate molecule was metabolised to the free form of DHE with serum concentrations in blood averaging 98.6 μM 15 mins post i.p. injection. The drug was rapidly excreted at a rate of 62 μM/hr with serum levels lowering to 12 μM 1 hr post administration. Total concentrations of DHE (conjugated +free) reached 120 μM 15 mins post administration and was excreted (120 μM/hr) reaching a serum concentration of 30.85 1 hr post administration (Table 1 and FIG. 1).

TABLE 1
Bisphosphate: free vs total

| | Average (μM) | | Free:Total |
|---|---|---|---|
| Time (hr) | Free | Total | ratio |
| 0.25 | 74.39 | 120.79 | 1.62 |
| 0.50 | 32.68 | 63.87 | 1.95 |
| 1 | 12.00 | 30.85 | 2.57 |
| 4 | 0.03 | 1.36 | 40.72 |
| 24 | 0.00 | 0.00 | 0.00 |

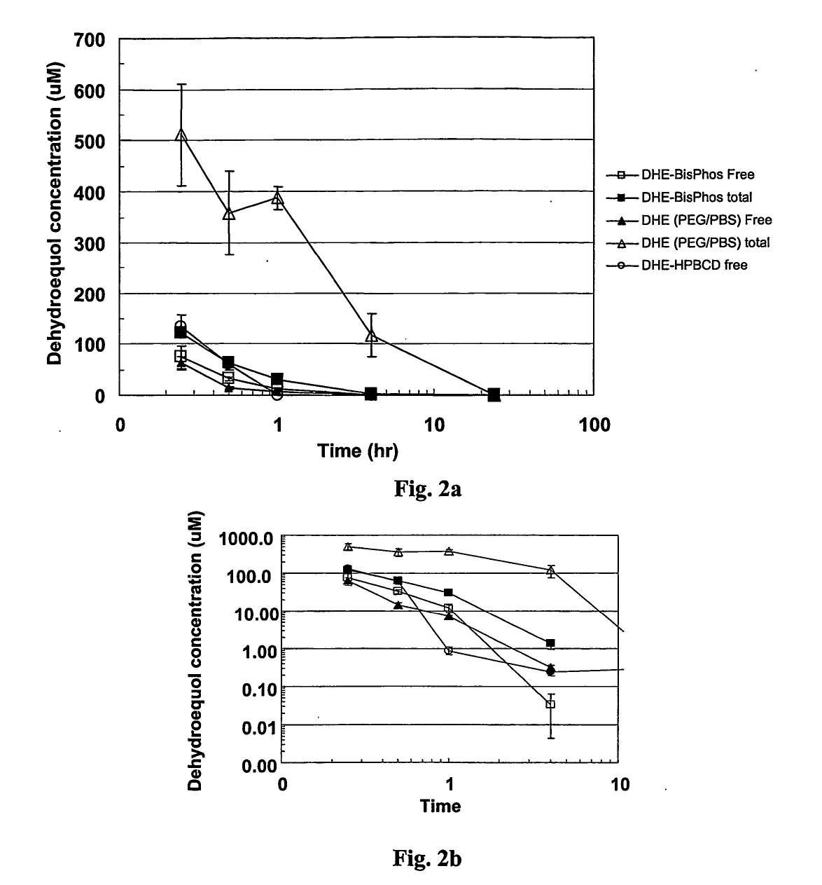

Comparison of free and total dehydroequol concentrations in sera taken from mice dosed i.p. with DHE-bisphosphate and a DHE-PEG:PBS formulation revealed approximately equal concentrations of the free form of the drug was achieved in serum 15 min post administration however, half the dosage of DHE-bisphosphate (25 mg/kg) was required to achieve this result compared with the DHE-PEG:PBS formulation (50 mg/kg) (74.4 μM vs 62 uM respectively) (Table 1, Table 2 and FIG. 2a). Interestingly, the observed free:total ratios for the DHE-bisphosphate preparation and DHE PEG:PBS formulation were some 5-fold different with more total DHE appearing in the plasma 15 min post-administration from rats dosed with the PEG:PBS formulation when compared to the DHE-bisphosphate preparation (120.8 μM phenoxodiol vs 511.6 μM dehydroequol). Plasma concentrations of free dehydroequol were some 1.8 fold (DHE-bisphosphate) and 2.2 fold (DHE-PEG:PBS) lower than those achieved in mice 15 min post administration of a HPBCD formulation of dehydroequol (50 mg/kg) (FIG. 2b; Table 3).

TABLE 2
PEG:PBS free vs total

| | Average (μM) | | Free:Total |
|---|---|---|---|
| Time (hr) | Free | Total | Ratio |
| 0.25 | 62.19 | 511.57 | 8.23 |
| 0.5 | 14.30 | 357.09 | 24.97 |
| 1 | 7.11 | 387.67 | 54.55 |
| 4 | 0.32 | 117.69 | 366.07 |
| 24 | 0.00 | 0.13 | 0.00 |

TABLE 3
Free DHE HPBCD formulation

| | Free serum PXD |
|---|---|
| | (μM) |
| Time (hr) | HPBCD |
| 0.25 | 134.02 |
| 0.50 | 61.44 |
| 1.00 | 0.88 |
| 4.00 | 0.24 |
| 24.00 | 0.32 |

Uses of esterified isoflavonoids include any presently known or later discovered uses for isoflavonoids or derivatives thereof including those listed above or described in the literature. The esterified isoflavonoids are found to be indicated in the treatment of osteoporosis and other symptoms of estrogen deficiency in postmenopausal women. Also, the compounds of the present invention are used to prevent osteoporosis and consequent fractures that result from osteoporosis, which are major contributors to morbidity and mortality in the elderly. Still further, the esterified isoflavones are used prophylactically to provide UV protection and in other ways to improve general skin health, to stimulate the immune system, and to reduce undesirable effects of oxidation (i.e., provide antioxidant benefits). Importantly the compounds of the invention are used to treat cancer, including breast, ovarian and prostrate cancers.

The isoflavonoid phosphate esters of the invention quite unexpectedly show some beneficial and/or marked activity in the subjects being treated. This comparison shows the particular utility and effectiveness of conjugated isoflavonoid compounds of the invention, and in particular those conjugates from compounds 1 to 34 described above.

Genistein phosphates are found to have poorer pharmacokinetic properties and profiles compared to the isoflavonoid counterparts described and exemplified above.

Thus, specific embodiments and applications of esterified isoflavonoid compounds have been disclosed. It should be apparent, however, to those skilled in the art that many more modifications besides those already described are possible without departing from the inventive concepts herein. The inventive subject matter, therefore, is not to be restricted except in the spirit of the appended claims.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification individually or collectively, and any and all combinations of any two or more of said steps or features.

The reference to any prior art in this specification is not, and should not be taken as, an acknowledgment or any form of suggestion that that prior art forms part of the common general knowledge in the field of endeavour.

1. An isoflavonoid phosphate ester compound of the general formula I: 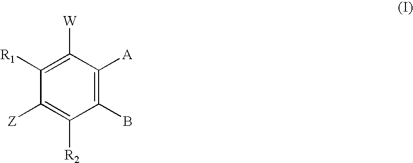
in which
R₁ R₂ and Z are independently M₂PO₄—, hydrogen, hydroxy, OR₉, OC(O)R₁₀, OS(O)R₁₀, CHO, C(O)R₁₀, COOH, CO₂R₁₀, CONR₃R₄, alkyl, haloalkyl, arylalkyl. alkenyl. alkynyl, aryl, heteroaryl, alkylaryl, alkoxyaryl, thio, alkylthio, amino, alkylamino, dialkylamino, nitro or halo, or
R₂ is as previously defined, and R₁ and Z taken together with the carbon atoms to which they are attached form a five-membered ring selected from 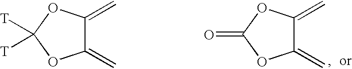
R₁ is as previously defined, and R₂ and Z taken together with the carbon atoms to which they are attached form a five-membered ring selected from 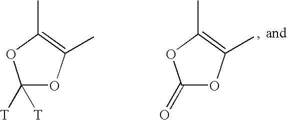
W is R₁, and A and B taken together with the carbon atoms to which they are attached form a six-membered ring selected from one of 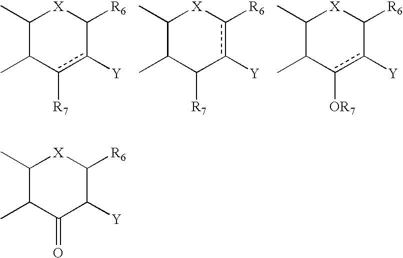
wherein
R₃ is hydrogen, alkyl, aryl, arylalkyl, an amino acid, C(O)R₁₁ where R₁₁, is hydrogen alkyl, aryl, arylalkyl or an amino acid, or CO₂R₁₂ where R₁₂ is hydrogen, alkyl, haloalkyl, aryl, heteroaryl or arylalkyl,
R₄ is hydrogen, alkyl or aryl,
or R₃ and R₄ taken together with the nitrogen to which they are attached comprise pyrrolidinyl or piperidinyl,
R₅ is M₂PO₄—, hydrogen, C(O)R₁₁ where R₁₁ is as previously defined, or CO₂R₁₂ where R₁₂ is as previously defined,
R₆, is M₂PO₄—, hydrogen, hydroxy, alkyl, aryl, amino, thio, NR₃R₄, COR₁₁ where R₁₁; is as previously defined, CO₂R₁₂ where R₁₂ is as previously defined or CONR₃R₄,
R₇ is hydrogen, C(O)R₁₁ where R₁₁ is as previously defined, alkyl, haloalkyl, aryl, arylalkyl or Si(R₁₃)₃ where each R₁₃ is independently hydrogen, alkyl or aryl, R₈ is M₂PO₄—, hydrogen, hydroxy, alkoxy or alkyl,
R₉ is alkyl, haloalkyl, aryl, arylalkyl, C(O)R₁₁ where R₁₁ is as previously defined, or Si(R₁₃)₃ where R₁₃ is as previously defined,
R₁₀, is hydrogen, alkyl, haloalkyl, amino, aryl, arylalkyl, an amino acid, alkylamino or dialkylamino,
the drawing “ [symbol] ” represents either a single bond or a double bond,
M is independently hydrogen, a straight or branched alkyl, alkenyl, alkenyl, alkoxyalkyl, alkvlthioalkyl, or aminoalkyl, a substituted or non-substituted cycloalkyl, an aryl, aralkyl, or alkylaryl, and a substituted cycloalkyl where at least one ring contains one or more of a nitrogen, sulfur, oxygen, phoshorous or silicon heteroatom in the at least one rng;
T is independently hydrogen, alkyl or aryl,
X is 0, NR₄ or S, preferably O, and
Y is 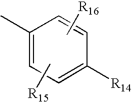
wherein
R₁₄, R₁₅ and R₁₆ are independently M₂PO₄—, hydrogen, hydroxy, OR₉, OC(O)R₁₀, OS(O)R₁₀CHO, C(O)R₁₀, COOH, CO₂R₁₀, CONR₃R₄, alkyl, haloalkyl, arylalkyl, alkenyl, alkynyl, aryl, heteroaryl, alkylaryl, alkoxyaryl, thio, alkylthio, amino, alkylamino, dialkylamino, nitro or halo,
and
wherein at least one of R₁, R₂, R₅, R₆, R₈, R₁₄, R₁₅, R₁₆, Z, W or A where present is independently MPO₄—,
or a pharmaceutically acceptable salt thereof,
with the proviso that compounds of the formula: 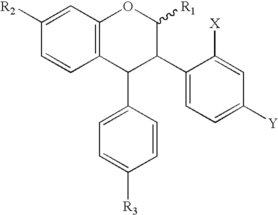
in which
R₁ is hydrogen or alkyl of 1-3 carbon atoms,
R₂ and R₃ are independently —OPO₃H₂, hydrogen or a substituent and at least one of R₂ and R₃ is —OPO₃H₂, and
X and Y are independently hydrogen or F,
are specifically excluded.

2. An isoflavonoid phosphate ester according to claim 1 of the general formula II: 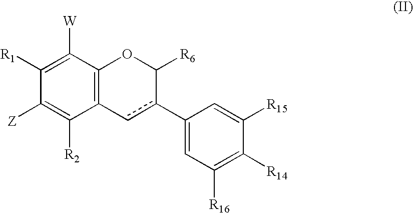
in which
R₁, R₂, R₅, R₆, R₁₄, R₁₅, W and Z are as defined in claim 1, and
the drawing “ [symbol] ” represents either a single bond or a double bond.

3. An isoflavonoid phosphate ester according to claim 2, wherein the drawing “ [symbol] ” represents a double bond.

4. An isoflavonoid phosphate ester according to claim 1 of the general formula III: 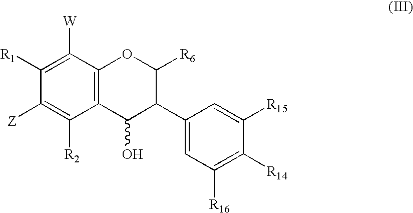
in which
R₁, R₂, R₅, R₆, R₁₄, R₁₅, W and Z are as defined in ciaim 1.

5. An isoflavonoid phosphate ester according to claim 1 of the general formula IV: 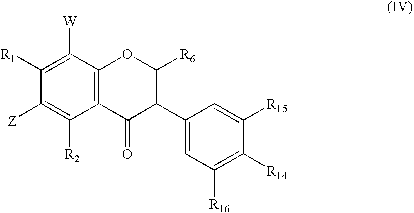
in which
R₁, R₂, R₅, R₆, R₁₄, R₁₅, W and Z are as defined in claim 1.

6. An isoflavonoid compound of claim 1, wherein the phosphate ester moiety is present as a corresponding salt M₂PO₄—, where M is a pharmaceutically acceptable cation.

7. An isoflavonoid compound of claim 6, wherein the corresponding salt M₂PO₄— is NaO(HO)P(O)O—.

8. An isoflavonoid compound selected from 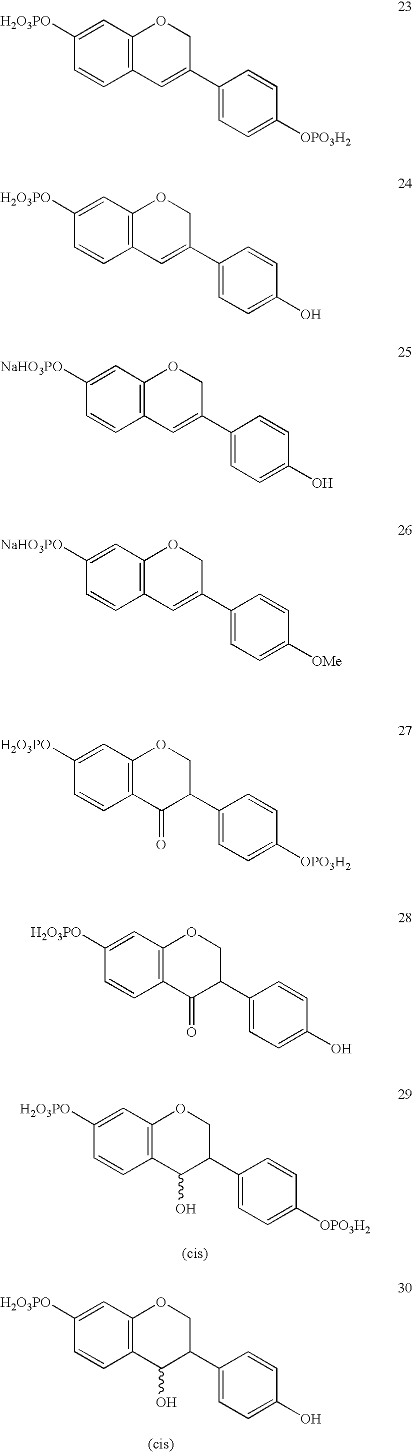 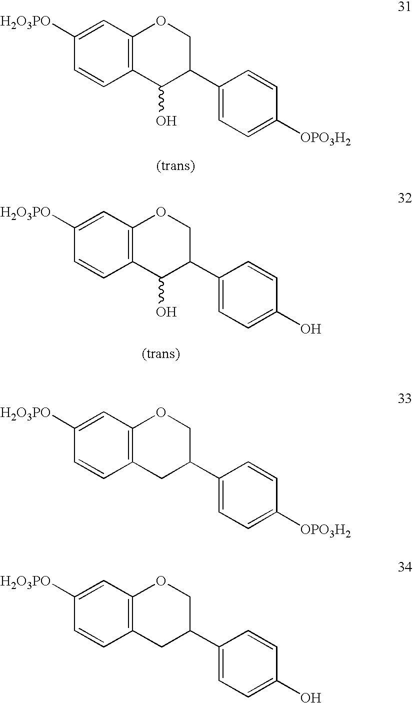
or a pharmaceutically acceptable salt thereof.

9. A method for the treatment, prophylaxis, amelioration, defence against, and/or prevention of one or more of the therapeutic indications as hereinbefore defined, which comprises administering to a subject a therapeutically effective amount of one or more compounds of formula I as defined in claim 1.

10. (canceled)

11. An agent for the treatment, prophylaxis, amelioration, defence against and/or treatment of one or more of the therapeutic indications as hereinbefore defined which comprises one or more compounds of formula I as defined in claim 1 either alone or in association with one or more carriers or excipients.

12. A method for the treatment, prophylaxis, amelioration, defence against and/or prevention of conditions in a subject associated with abnormal estrogen/androgen balance which method includes the step of administering one or more compounds of formula I as defined in claim 1 either alone or in association with one or more carriers or excipients.

13. A method of claim 12, wherein the subject is a woman.

14. A method of claim 12, wherein the subject is a man.

15. (canceled)

16. A therapeutic composition which comprises one or more compounds of formula I as defined in claim 1 in association with one or more pharmaceutical carriers and/or excipients.

17. A drink or food-stuff, which contains one or more compounds of formula I as defined in claim 1.

18. A composition comprising one or more compounds of formula I as defined in claim 1, vitamin E, and optionally pharmaceutically, veterinarily or cosmetically acceptable carriers and/or excipients.

19. (canceled)
